(19) United States
(12) Patent Application Publication
(10) Pub. No.: US 2020/0306477 A1
(43) Pub. Date: Oct. 1, 2020

(54) Bougie with Improved Sensory Feed

(71) Applicant: THE TRUSTEES OF THE STEVENS INSTITUTE OF TECHNOLOGY, HOBOKEN, NJ (US)

(72) Inventors: Bailey Keith Bancroft, Harleysville, PA (US); Jessica Rose Martin, Staten Island, NY (US); Cari Schoffelman, Hoboken, NJ (US); Zach Wilhelm, Tuckerton, NJ (US); Vicki Hazelwood, Wayne, NJ (US); Valerie DeAngelo, Staten Island, NY (US); David Zodda, Montclair, NJ (US)

(73) Assignee: THE TRUSTEES OF THE STEVENS INSTITUTE OF TECHNOLOGY, HOBOKEN, NJ (US)

(21) Appl. No.: 16/831,697

(22) Filed: Mar. 26, 2020

Related U.S. Application Data
(60) Provisional application No. 62/824,993, filed on Mar. 27, 2019.

(51) Int. Cl. A61M 16/04

(52) U.S. Cl. CPC A61M 16/0488; A61M 2205/587; A61M 2205/8206; A61M 2205/583; A61M 2205/582

(57) ABSTRACT

An improved bougie provides two-fold verification of proper placement of an endotracheal tube during medical intubation. Features for both tactile feedback, via a flexible body with a coude tip, and visual feedback, via a transilluminating light source, are provided. The improved bougie is fully disposable.

### CROSS-REFERENCE TO RELATED APPLICATIONS

This application claims priority to U.S. provisional application Ser. No. 62/824,993, filed on Mar. 27, 2019, the entire contents of which are incorporated herein by reference.

### FIELD OF THE INVENTION

The present invention relates to the field of medical devices, and more particularly, to medical instruments used for the introduction of endotracheal intubation means.

### BACKGROUND OF THE INVENTION

Emergency intubations are performed on 1.8 million patients per year. In pressure situations, it is crucial to intubate a patient successfully (i.e., down the trachea and not the esophagus). Statistics reveal that as much as 25% of all emergency intubations require multiple attempts. Failed intubations (e.g., esophageal intubation) can result in complications, including, for example, cardiac arrest and permanent brain damage. Physicians therefore need verification and assurance that they are intubating the trachea and not the esophagus.

Bougies are thin cylinders of rubber, plastic, metal, or another material that a physician inserts into or through a body passageway, such as the trachea, to diagnose or treat a condition. A bougie may be used to guide another instrument into a passageway. Bougies are available in a wide range of sizes and degrees of flexibility. The cylinder may be equipped with a light source. Traditionally, bougies have been used to guide and properly place an endotracheal tube (ET tube) that is adapted to intubate a patient for the purpose of delivering oxygen, anesthesia, medicine, etc. thereto. The provision of rrcoude (i.e., angled) tips on bougies is a common approach that facilitates the guidance and proper placement of bougies.

The guidance and proper placement of bougies has also been aided by providing them with a light source. Such lighted bougies can project light through the skin and tissue of the throat/trachea to provide visual verification of their placement, which is also known as transillumination.

Although the aforementioned techniques (i.e., coude tips and light sources) provide some verification of the correct bougie placement, they do not provide the bougies with a simple way to determine their proper angular orientation within a patient's throat. This is especially the case with bougies of uniform, circular cross-sections. In addition to producing heat and/or having exposed glass that can hurt a patient, lighted instruments can sometimes be unreliable, depending on the condition of the patient, especially if a trauma patient. Overall, conventional bougies, whether lighted or not, are only approximately 50% accurate in determining proper bougie placement.

### SUMMARY OF THE INVENTION

The present invention relates to an intubation device that is flexible and that incorporates a transilluminating light source in combination with a coude tip. The light source provides transillumination, while the coude tip provides tactile feedback; thus, the present invention provides twofold verification of correct intubation, thereby enabling quicker and more efficient intubation through reduction of the number of second attempts necessitated after an initial, unsuccessful endotracheal intubation.

In an embodiment, the intubation device of the present invention has a housing unit and a bougie, both of which are fully disposable. The bougie is flexible and is used to probe the trachea. The bougie has a light source at one end for transillumination purposes. Because an LED is used, the light source does not generate a harmful level of heat. The bougie, in turn, is connected to a housing unit at the end (i.e., the proximal end) opposite to the light source, thereby connecting the light source to a battery housed within the housing unit's battery compartment. To form the circuit, the battery, LED and a resistor are soldered together on a circuit board. Additionally, it should be noted that the housing unit can also function as a handle for the intubation device.

It has been demonstrated that the light source of the present invention can illuminate through the range of human trachea and tissue of interest, with thicknesses of from about 0.6 cm to about 3.65 cm.

In some embodiments, the placement of notches, ridges, nubs, etc. on the proximal end of the bougie provides a tactile frame of reference for ensuring proper rotational orientation of the bougie, which supplements other forms of feedback, such as those provided by light and coude tip embodiments. This mechanism facilitates straightforward intubation regardless of the position in which the patient enters the emergency room.

In certain embodiments, the body of the bougie, either the entirety of the body or a portion thereof, can have a non-circular cross-section (e.g., trapezoidal or rectangular) in order to indicate at a glance which end of the bougie is proximal and to indicate the rotational orientation of the bougie.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a more complete understanding of the present disclosure, reference is made to the following detailed description of various exemplary embodiments considered in conjunction with the accompanying drawings, in which:

FIG. 1 is an exploded perspective view of a medical instrument having two-fold verification and constructed in conformance with one embodiment of the present invention;

FIG. 2 is a perspective view of a medical instrument having three-fold verification and constructed in conformance with another embodiment of the present invention;

FIG. 3 is a perspective view of a medical instrument having a plurality of light sources and constructed in conformance with a further embodiment of the present invention;

FIG. 4 is a flow chart representing two methods of using the medical instruments illustrated in FIGS. 1-3 in connection with an intubation procedure;

FIG. 5 is an illustration of how one embodiment of the present invention (i.e., a bougie having a light-emitting coude tip at its distal end) may be used to intubate a patient;

FIG. 6 is an illustration which depicts a bougie-type device which is opaque except for a transparent window at its distal end through which light from an LED is emitted;

FIGS. 7A-7C are a series of views (i.e., bottom, top and end, respectively) of a bougie in accordance with an embodiment of the present invention, schematically depicting a bougie-type device with transparent windows on its bottom and at its distal end;

FIG. 8 is an illustration of how yet another embodiment of the present invention (i.e., a bougie adapted to emit light along the length of a superior side of the device, as well as from a coude tip located at a distal end of the device) may be used to intubate a patient;

FIG. 9 is an illustration of how a still further embodiment of the present invention (i.e., a bougie adapted to emit light from all sides of the device, as well as from a coude tip located at a distal end of the device) may be used to intubate a patient;

FIG. 10 is a schematic representation depicting a situation in which light is only emitted from the top and/or bottom of a bougie constructed in conformance with the present invention;

FIGS. 11A and 11B are schematic representations depicting a situation in which light is not emitted from a proximal end of a bougie constructed in conformance with the present invention, but is selectively emitted from the bougie's distal end;

FIG. 12 is a sketch depicting a bougie which, in conformance with an embodiment of the present invention, is provided with a flat superior surface along the entire length of the bougie;

FIG. 13 is a sketch depicting a bougie which, in conformance with an embodiment of the present invention, is provided with a flat surface exclusively at the proximal end of the bougie;

FIG. 14 is a sketch depicting a bougie which, in conformance with an embodiment of the present invention, is provided with tactile means (e.g., a plurality of dimples) at the proximal end of the bougie; and

FIG. 15 is a sketch depicting a bougie which, in conformance with an embodiment of the present invention, is provided with drug or oxygen delivery means in the form of a hollow central region (e.g., a channel or passageway).

### DETAILED DESCRIPTION OF EXEMPLARY EMBODIMENTS

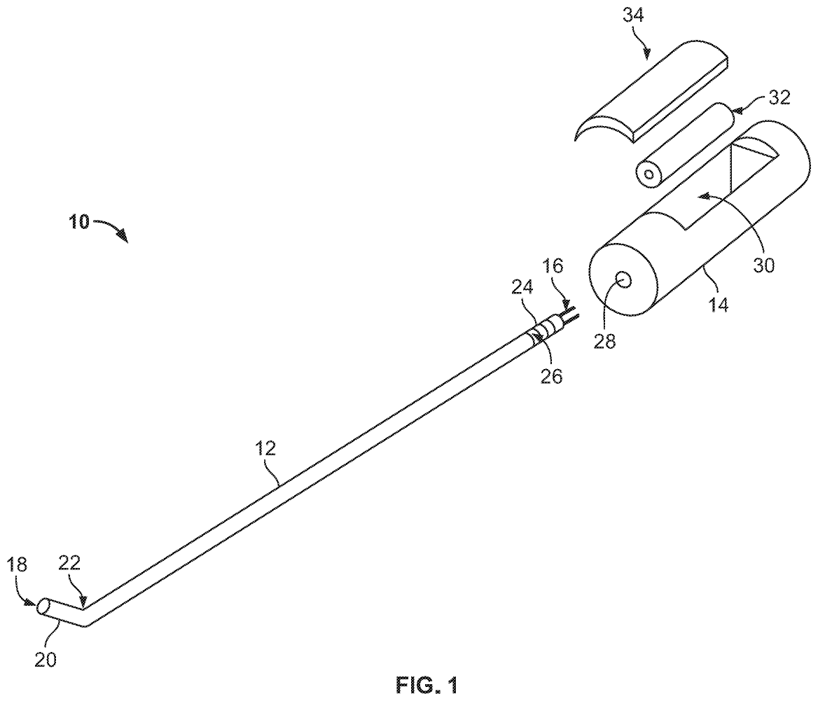

With reference now to FIG. 1, a device for endotracheal intubation 10 is displayed, divided into two major components: a bougie 12 and a housing 14. Both bougie 12 and housing 14 are disposable, adding to the device's ease of use. The bougie 12 is flexible and is used to probe the patient's trachea (not shown) and verify placement of and/or guide the endotracheal tube (not shown). Bougie 12 has conductive means (e.g., wires) 16 running through it to provide power. In alternate embodiments, the conductive means 16 can comprise one or more fiber optic cables running through the length of bougie 12. In some embodiments, such a fiber optic cable can be side-emitting, or only end-emitting.

The conductive means 16 functions to provide power to an LED 18 at a distal end 20 of bougie 12. The distal end 20 is angled such that it forms a coude tip 22, for improved tactile and auditory feedback. The proximal end 24 of the bougie includes a male-threaded end 26 that interfaces with a female-threaded aperture 28 of housing 14, allowing the bougie 12 and housing 14 to removably interlock. Housing 14 further comprises a battery compartment 30 containing a battery 32 and a battery cover 34 to secure the battery 32 within the battery compartment 30. In some embodiments, LED 18 is located at the proximal end 24 of the bougie 12, so as to avoid putting LED 18 physically inside the patient.

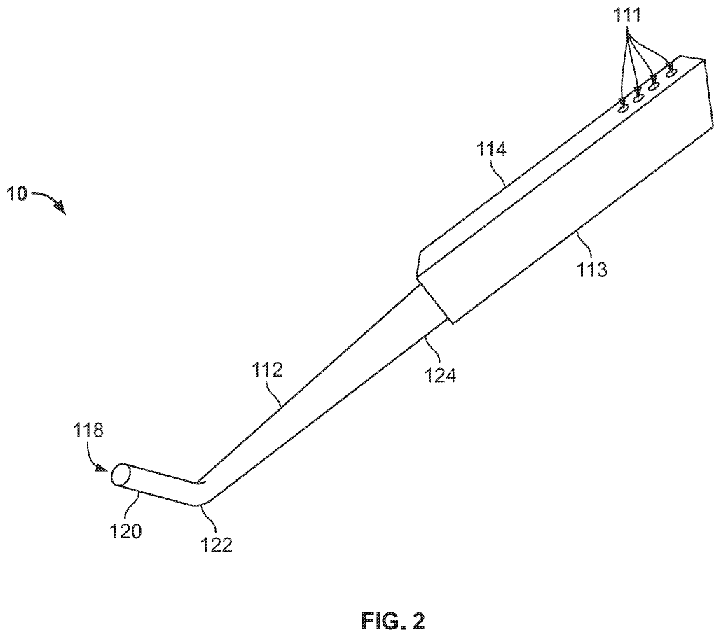

Turning now to FIG. 2, device 110 includes tactile indicators 111, which can be notches, nubs, bumps, ridges, dimples, or anything known in the art having equivalent functionality. In an embodiment, the device 110 has a non-circular cross-sectional shape 113. This cross-sectional shape 113 can, among others, be trapezoidal or rectangular, serving the function of distinguishing, through visual or tactile means, the proximal end 124 of the bougie 112 from its distal end 120. In an embodiment, the non-circular cross-sectional shape 113 is only present at the proximal end 118. In other embodiments, the non-circular cross-sectional shape 113 can extend partly down bougie 112, or even all the way down to coude tip 122.

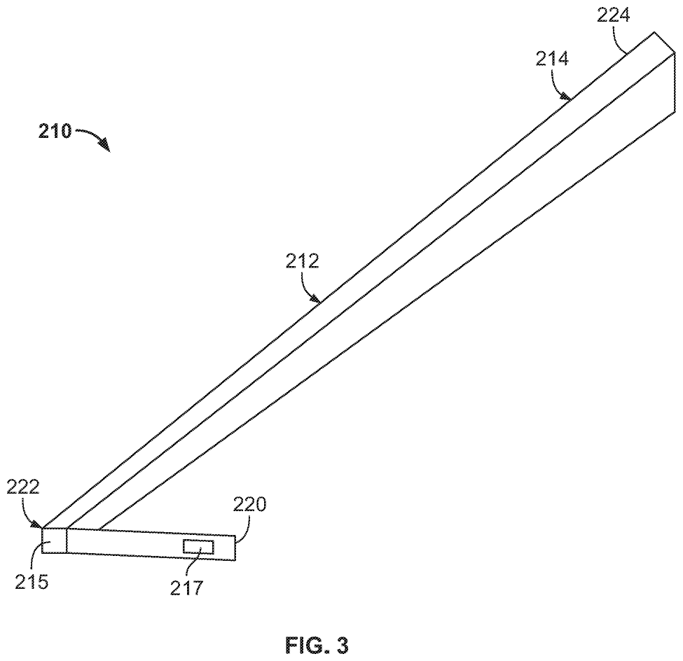

Turning now to FIG. 3, conductive means (not shown) functions to provide power to light sources 215, 217 at a distal end 220 of bougie 212. Specifically, FIG. 3 shows that distal end 220 contains a plurality of light sources 215, 217. Collectively, the light sources 215, 217 function to emit light at the distal end 220 such that the light projects both through the neck (not shown) of the patient, as well as from near coude tip 222, such that the light faces back towards the trachea (not shown).

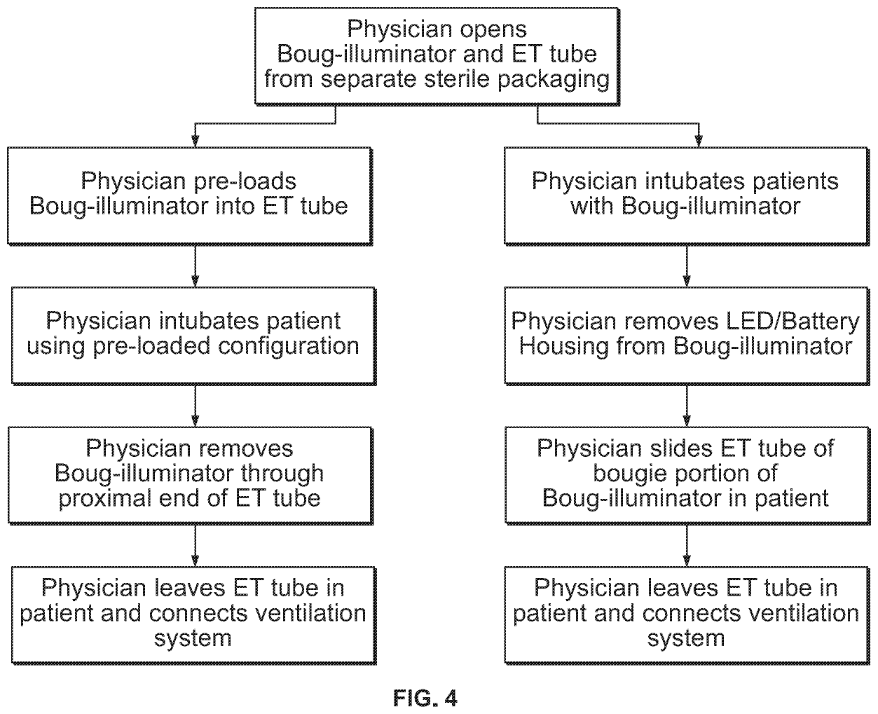

Turning now to FIG. 4, in operation, a physician can execute the procedure of pre-loading the bougie of the present invention in a conventional endotracheal tube, intubating the patient with this configuration, verifying correct placement of the ET tube via transillumination and/or audio or tactile feedback, removing the bougie while leaving the ET tube in the patient, and then connecting the ET tube to a ventilation system.

In another embodiment, the patient may be intubated directly using the bougie of the present invention. First, the physician places the bougie in the patient's trachea, using the confirmation methods (tactile feedback, coude tip, transillumination). Once correct placement is confirmed, the physician removes the bougie's housing (e.g., by undoing a snap fit mechanism) and an endotracheal tube connected to a ventilation system can be inserted over the remainder of the device to deliver air and/or medicine to the patient. The bougie can then be removed while leaving the ET tube in place.

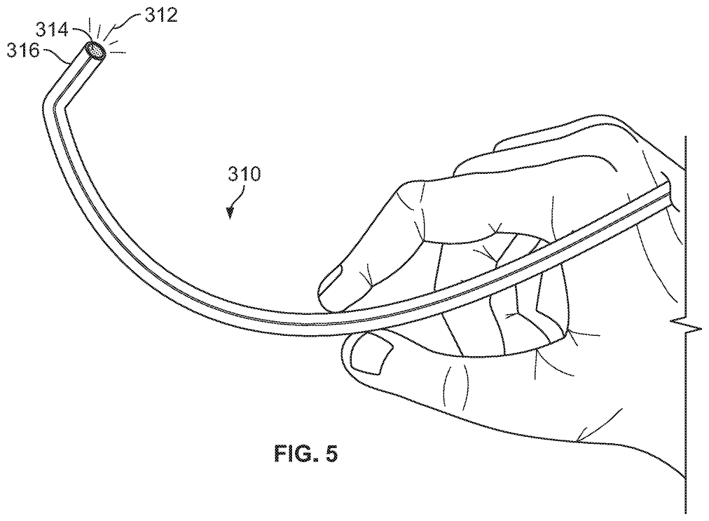

Turning now to FIG. 5, a distal light bougie 310 is shown. A light source, such as an LED (not shown) located at either the proximal or distal end of the bougie, emits light 312 through a distal end 314 of the bougie's coude tip 316 to provide transillumination.

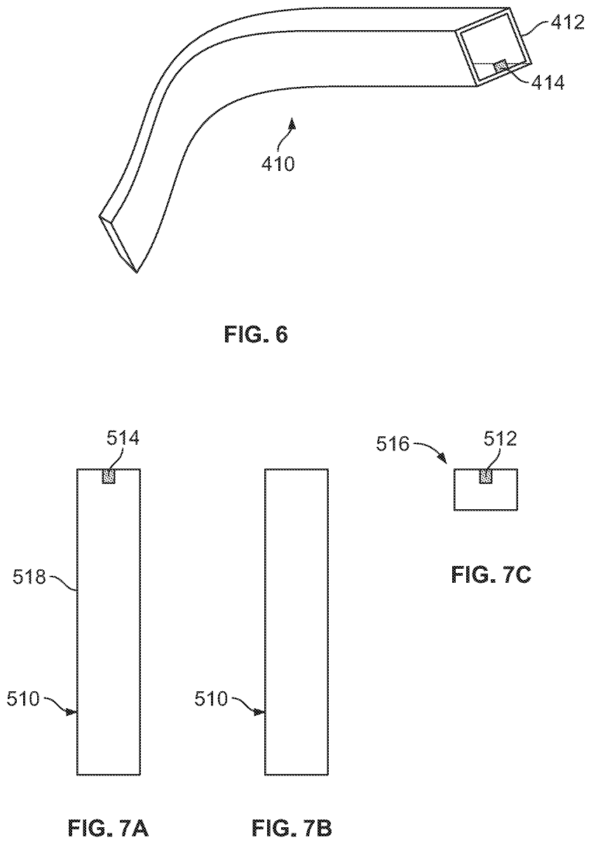

With respect to FIG. 6, a bougie 410 with a windowed tip 412 is shown. Light exits through a window 414 for more focused transillumination; the remainder of the bougie 410 is opaque.

Referring now to FIGS. 7A-7C, an alternate embodiment of the present invention is shown in which the bougie 510 has two windows 512, 514. One window 512 allows light to exit at a tip 516 of the bougie 510; the other window 514 allows light to exit a bottom surface 518 of the bougie 510.

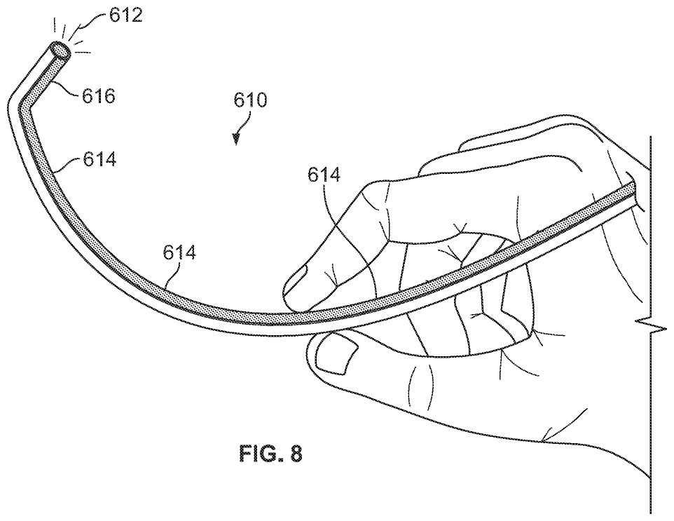

Referring now to FIG. 8, a coude-tip bougie 610 configured to shine light 612 through the entire length of a superior surface 614 of the bougie and coude tip 616 (i.e., the surfaces on the inside of the coude tip's bend is shown). Such a configuration provides transillumination and rotational orientation information due to the resulting selective illumination.

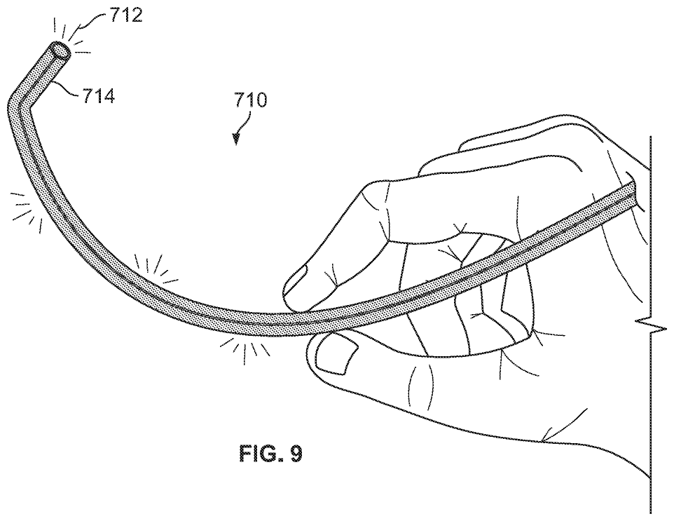

With reference to FIG. 9, a coude-tip bougie 710 configured to shine light 712 through the entirety of the bougie 710 and coude tip 714 (i.e., the entire surface area) is shown. Such a configuration provides transillumination verification of proper intubation via the positioning of light penetrating through the patient's neck, irrespective of the orientation of the coude tip 714.

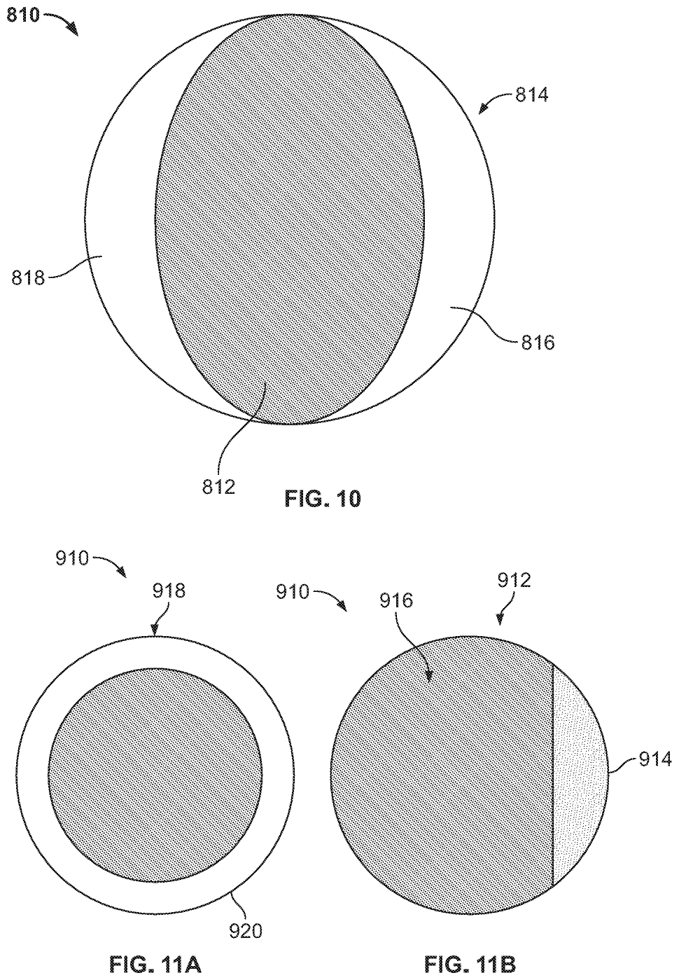

In some embodiments, the body of the bougie can have selective side-emitting properties. For instance, in FIG. 10, the bougie 810 emits light 812 (represented by a shaded oval) from the top and the bottom, but is blocked (represented by unshaded portion 814) from emitting light from its right and left sides 816, 818.

FIGS. 11A and 11B illustrates a bougie 910 having a tapered distal end 912 adapted to emit light from a side 914 and an end 916 of its distal end 912. A proximal end 918 of the bougie 910 is, however, blocked (represented by shaded portion 920) from side-emission of light.

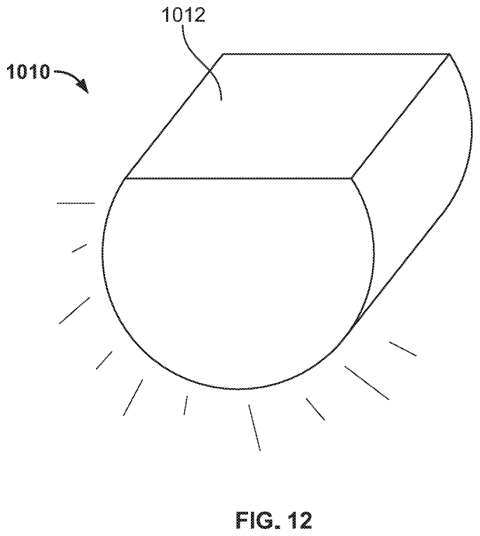

FIG. 12 shows a bougie 1010 made in accordance with an embodiment of the present invention. A superior surface 1012 of the bougie 1010 is flat, to provide visual and tactile indication of the coude tip's (not shown) rotational orientation.

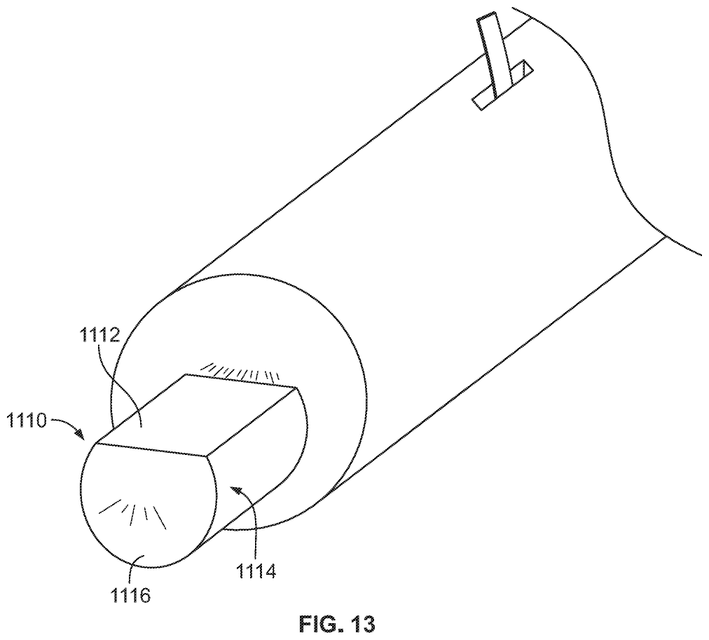

Referring now to FIG. 13, a bougie 1110 made in accordance with an embodiment of the present invention is shown. Like the embodiment of FIG. 12, the bougie of FIG. 13 has a superior surface 1112 that is flat to provide visual and tactile indication of the coude tip's (not shown) rotational orientation, whereby a proximal end 1114 of the bougie 1110 is provided with a non-circular cross-sectional shape 1116.

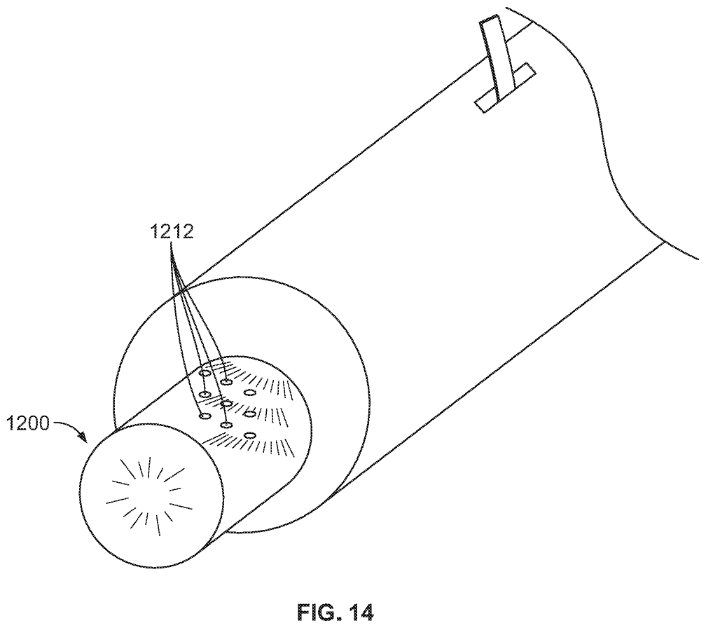

With reference to FIG. 14, a bougie 1210 with tactile indicia 1212 (i.e., bumps) on its proximal end is shown. The indicia or bumps 1212 provide visual and tactile information regarding the coude tip's (not shown) rotational orientation.

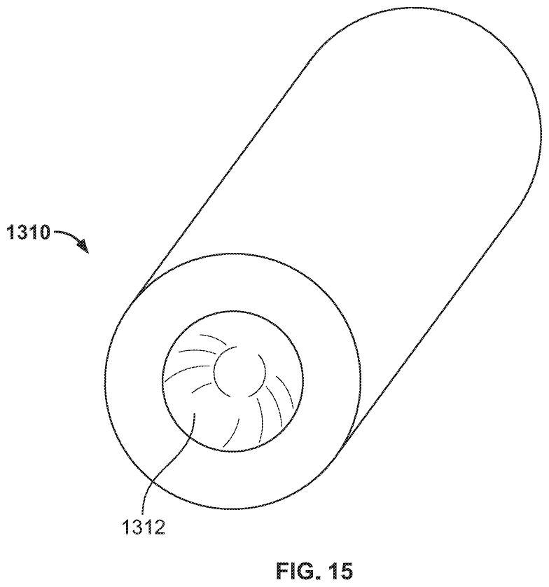

Referring now to FIG. 15, a bougie 1310 adapted to deliver oxygen and/or medicine to the trachea through its hollow central region 1312 is shown.

In a further embodiment, the bougie tapers along its length from its proximal end to its distal end (not shown).

In additional embodiments, the bougie lights up like a glow stick via a safe chemical reaction inside the lumen of the device, thereby eliminating the need for an external battery and an LED.

It will be understood that the embodiments described hereinabove are merely exemplary and that a person skilled in the art may make many variations, combinations, permutations and modifications thereof without departing from the spirit and scope of the present invention. All such variations and modifications are intended to be included within the scope of the present invention as it is explicitly and inherently described hereinabove and as illustrated in the accompanying drawings.

1. A bougie for medical intubation, comprising:
a flexible tubular body having a proximal end and a distal end, said distal end including a flexible tip;
a housing removably coupled to said proximal end of said body, said housing configured and arranged to be grippable by a user;
a light source integrated with at least one of said housing and said body, said light source configured and arranged to emit light from said body and thereby provide a user with a visible indication of the location of said bougie during the performance of a medical intubation; and
indicia provided on at least one of said body and said housing, said indicia configured and arranged to provide a user with a tactile indication of the rotational orientation of said bougie during the performance of a medical intubation.

2. The bougie of claim 1, wherein said housing and said body of said bougie are disposable following the performance of a medical intubation.

3. The bougie of claim 1, wherein said tip is a coude tip forming a bend at said distal end of said body, whereby said indicia provide a user with a tactile indication of the rotational orientation of said coude tip during the performance of a medical intubation.

4. The bougie of claim 3, wherein said light source comprises a light-emitting diode located at said coude tip of said body.

5. The bougie of claim 1, wherein said light source is adapted to provide transillumination through tissues of a patient's trachea, whereby emitted light is visible through a patient's neck during the performance of a medical intubation.

6. The bougie of claim 1, wherein said indicia comprise a flat surface arranged along at least a partial length of one side of said body, thereby allowing a user to tactilely distinguish said one side of said body from other sides of said body.

7. The bougie of claim 1, wherein said housing comprises a battery compartment housing a battery, said battery being electrically coupled to said light source via electrical conductors extending from said housing to said light source.

8. The bougie of claim 1, wherein said body is sufficiently translucent or transparent such that said light source can emit light through all sides of said body simultaneously during the performance of a medical intubation.

9. The bougie of claim 1, wherein one side of said body is sufficiently translucent or transparent such that said light source can emit light exclusively through said one side of said body.

10. The bougie of claim 9, wherein said light source is disposed near said tip of said body and configured and arranged to emit light through said tip.

11. The bougie of claim 1, wherein said body is opaque, said opaque body comprising one or more transparent or translucent windows through which light can be emitted.

12. The bougie of claim 11, wherein said distal end of said body includes a first window on said tip of said distal end, wherein said tip is an angled coude tip, and a second window on a bottom surface of said distal end, thereby providing light for illumination in at least two directions.

13. The bougie of claim 1, wherein said light source comprises light-producing chemical reactants housed within an internal cavity of said body, whereby said light source continuously emits light independent of an outside power source.

14. The bougie of claim 1, wherein said light source is disposed near said tip of said body and configured and arranged to emit light through said tip.

15. The bougie of claim 1, wherein said proximal end of said body has a first cross-sectional shape and said distal end of said body has a second cross-sectional shape, said second cross-sectional shape being visually and tactilely distinct from said first cross-sectional shape.

16. The bougie of claim 1, wherein said indicia comprise a tactilely distinct textured region on said housing.

17. The bougie of claim 1, wherein said body includes a hollow central conduit passing from said proximal end to said distal end, said conduit being configured and arranged to enable the delivery of oxygen or medicine therethrough.

18. A bougie for medical intubation, comprising:
a flexible tubular body having a proximal end and a distal end, said distal end including a flexible coude tip;
a housing removably coupled to said proximal end of said body, said housing configured to be grippable by a user;
a fiberoptic member extending from said housing to said distal end of said body;
a light source integrated with said housing, said light source configured and arranged to emit light through said tip via said fiberoptic member to thereby provide a user with a visible indication of the location of said bougie during the performance of a medical intubation; and
indicia provided on at least one of said body and said housing, said indicia configured to provide a user with a tactile indication of the rotational orientation of said bougie during the performance of a medical intubation.

19. A bougie for medical intubation, comprising:
a translucent or transparent, flexible tubular body, including a proximal end having a first cross-sectional shape, a distal end having a second cross-sectional shape visually and tactilely distinct from said first cross-sectional shape, a plurality of sides, including a flat surface arranged along at least a partial length of one side of said body, one or more transparent or translucent windows disposed on said plurality of sides through which light can be emitted, and a flexible, angled coude tip at said distal end of said body;
one or more light-emitting diodes disposed near said coude tip of said body and configured and arranged to emit light through said coude tip, said one or more light-emitting diodes configured to provide transillumination through a patient's trachea and thereby provide a user with a visible indication of where said bougie is located during the performance of a medical intubation;
a housing removably coupled to said proximal end of said body, said housing being configured to be grippable by a user and including a tactilely distinct textured region of indicia on said housing, said indicia configured to provide a user with feedback regarding a rotational orientation of said bougie and said coude tip during the performance of a medical intubation, and wherein said housing comprises a battery compartment housing a battery; and
electrical conductors, configured and arranged for electrically coupling said battery to said one or more light-emitting diodes.
